(19) United States
(12) Patent Application Publication
(10) Pub. No.: US 2014/0323814 A1
(43) Pub. Date: Oct. 30, 2014

(54) Method for Monitoring the Medical Condition of a Patient

(71) Applicant: Thomas Löser, Leipzig (DE)

(72) Inventor: Thomas Löser, Leipzig (DE)

(73) Assignee: Löser Medizintechnik GmbH, Leipzig (DE)

(21) Appl. No.: 13/810,610

(22) PCT Filed: Jul. 12, 2011
(86) PCT No.: PCT/DE2011/075162

§ 371 Date: Jul. 14, 2014

(30) Foreign Application Priority Data
Jul. 16, 2010 (DE) 10 2010 027 486.0

(51) Int. Cl. A61B 5/00

(52) U.S. Cl. CPC A61B 5/4845; A61B 5/7275
USPC 600/300

(57) ABSTRACT

The invention relates to a method for monitoring the medical condition of a patient by means of a patient data management and/or decision assist system. Here the following steps are provided:
(a) detecting values of physiological parameters of the patient, wherein at least a part of the physiological parameters characterizes the presence of a disturbance of health;
(b) linking the detected values to a first time stamp and a second time stamp, wherein the first time stamp indicates the time of occurrence of the value and the second time stamp the time of detection of the value;
(c) continuously comparing the values of different parameters having the same first time stamp with comparative values stored in the system;
(d) triggering an alert if the comparison shows that at least one of the values is outside of the given tolerance range with respect to the comparative value;
(e) linking the alert to a third time stamp indicating the time of triggering the alert;
(f) determining the time (final time) as from the third time stamp at which a medical measure must be taken at the latest, wherein the determination of the time is made on the basis of given time limits stored in the system; and
(g) continuously outputting (i) the final time determined in step (f) and/or (ii) the period left to reach the final time and/or (iii) a time domain being shorter than the period left to reach the final time.

The invention relates to a method for monitoring the medical condition of a patient as well as a system for carrying out this method.

In the public health system, in particular in hospitals, there are employed patient data management systems wherein the patient data are stored. In addition to the so called master data of the patient such as name, Christian name, and date of birth the patient data comprise the medical findings and initiated or already finished medical or caring measures. In the following such measures are also referred to as medical measures.

There are further known systems to assist the physician in the decision which medical measure in a given situation is the right one. For example, WO 2008/044189 A2 discloses a decision assist system intended for use in the treatment of a sepsis.

A sepsis is defined as “the entirety of life-threatening clinical symptoms and pathophysiological changes in response to the action of pathogenic germs and their products that invade the blood stream from an infective focus, activate the large biological cascade mechanisms and specific cell systems, and trigger formation and release of humoral and cellular mediators” (Schuster, H. P., and Müller-Werdan, U.: Definition und Diagnose von Sepsis und Multiorganversagen in Sepsis und MODS, Berlin 2005). If additionally an organ of the patient is acutely affected, then there is a severe sepsis.

Typical pathogens of a sepsis are Staphylococcus aureus, coagulase-negative staphylococci, enterococci, Escheria coli, Klebsiella spp., Enterbacter ssp., and Pseudomonas aeruginosa.

At the beginning of an intensive medical treatment, i.e. usually in the admission of a patient in the intensive care unit of a hospital, it is often not known whether the patient suffers from an infection or not. Well known is, however, which pathogens, in particular which germs, are already present in the intensive care unit. For this reason, in the course of a so called “calculated therapy” immediately at the beginning of the intensive medical treatment one or more medicaments are given to treat typical infections, in particular those infections caused by the germs known to be present in the unit. Such medicaments include, e.g. antibiotics, virostatics, and antimycotics, wherein the calculated therapy typically includes the administration of an antibiotic.

At the same time, at the beginning of the intensive medical treatment there is taken a sample from the patient that is tested for pathogenic germs. The test that is connected for example with the detection of antibodies or the genetic information of pathogens (e.g. species PCR) or the culturing of cell cultures is performed in specialized laboratories. The results of this test are typically available after three days. The results show what pathogenic germs and resistances are present in the patient. As soon as this is known the medical treatment is adapted to the actual condition of the patient.

The data stored in the patient data management systems are entered into the system via standardized interfaces (in hospitals HL7, in practices xDT) by the persons treating the patient as well as by the laboratory personnel and on request placed at the disposal of the treating person.

In particular, with multimorbid patients the number of data that have to be detected for each individual patient by the patient data management system is extremely high. However, due to the considerable medical advance at present with supposedly simple diseases a large number of data are detected that often do not allow a proper analysis. Thus, it is not atypical that medical decisions are made on the basis of single data that are considered to be particularly conspicuous. Other data stored in the patient data management system that could support an other finding are ignored, so that due to rather arbitrarily selected data medical measures are taken that in complete analysis of all data could turn out to be false or insufficient.

Moreover, it is a widespread problem that indeed not all of the patient's data that should be detected indeed are detected. Nevertheless, it is often not evident to the responsible physicians and nurses that there are data missing.

On the other hand, in practice there are often detected data that have no use for the diagnosis and therapy of a patient's disease. For example, according to rigid schemes there are requested laboratory values, wherein depending on the value regulations are made for example daily or weekly. Such measurements are time-consuming and costly and may put additional strain on the patient.

A further serious problem is that certain data come into the patient data management system at a time at which they are no longer up-to-date. This has the consequence that there is attached an importance to the data which would not fall to them in the correct chronological arrangement. This is particularly disadvantageous if data stored in the patient data management system are analyzed according to given criteria and alerts are triggered on the basis of this analysis.

In triggering an alert it often does not receive the appropriate attention in clinical practice since the number of alertings, for example in an intensive care facility is such often that not every alert can be immediately noted. Thus, between alerting and initiating the required medical measure there passes time which in the worst case may fully or partially impair the success of the treatment. Moreover, also even if a skilled person immediately takes note of the alert in most cases it is not readily clear which medical measure is to be taken. Thus, the high number of collected data requires weighting of the data. Here, classification methods can be useful, as described for example in EP 1 687 756 A1. Finally, a decision assist system can be based on a classification method that should lead the physician to an objective diagnosis. For example, such decision assist systems are based on guidelines issued by medical societies, in case of sepsis the guidelines of the Surviving Sepsis Campaign (SSC), for example.

All these factors in particular in the field of intensive care lead to an error cascade that can be triggered in the treatment of a patient: the data are incomplete; the existing data have a wrong chronological arrangement; alerting is noticed too late, data are wrongly weighted, thus, the diagnosis is wrong, or, if right at all, is made late.

Object of the invention is to eliminate the disadvantages of the prior art. In particular, a method for monitoring the medical condition of a patient should be indicated which enables an objective medical treatment which can also be associated with a significant reduction in the treating time of the patient. Further, a system for carrying out this method should be indicated.

This object is solved by the features of claims 1 and 8. Suitable developments of the inventions result from the features of claims 2 to 7.

In accordance with the invention there is provided a method for monitoring the medical condition of a patient by means of a patient data management and/or decision assist system comprising the steps of
(a) detecting values of physiological parameters of the patient, wherein at least a part of the physiological parameters characterizes the presence of a disturbance of health;
(b) linking the detected values to a first time stamp and a second time stamp, wherein the first time stamp indicates the time of occurrence of the value and the second time stamp the time of detection of the value;
(c) continuously comparing the values of different parameters having the same first time stamp with comparative values stored in the system;
(d) triggering an alert if the comparison shows that at least one of the values is outside of the given tolerance range with respect to the comparative value;
(e) linking the alert to a third time stamp indicating the time of triggering the alert;
(f) determining the time (final time) as from the third time stamp at which a medical measure must be taken at the latest, wherein the determination of the time is made on the basis of given time limits stored in the system; and
(g) continuously outputting (i) the final time determined in step (f) and/or (ii) the period left to reach the final time and/or (iii) a time domain being shorter than the period left to reach the final time.

The term “time of occurrence of the value”, in the following also referred to as “occurrence time”, relates to the time of occurrence at which the value of a parameter actually occurred in a patient. This time is independent of when the value is actually determined. This time is also independent of when the value is actually detected in the system. If sampling is required, as is the case with parameters that must be determined in laboratory, for example then the time of sampling is the occurrence time.

The term “time of detection of the value”, in the following also referred to as “detection time” relates to the time at which the value reaches the system. The system can automatically assign the detection time to each value.

By linking each value to two time stamps it is ensured that in step (c) there are only considered values actually having the same first time stamp. Thus, it is ensured that in the analysis of the data actually only data having the same occurrence time are taken into account. This is particularly important if a significant difference in time is between occurrence time and the detection time. For example, with laboratory values there can be three days between the occurrence time and the detection time.

The time stamp is a value in a given format which assigns an occurrence to a specific time. For example, the time stamp can code day, date, year, hour, minute, and second of the time of the occurrence. To simplify the comparison in step (c) a tolerance value can be given.

Said tolerance value may be for example 2 s, 5 s, 10 s, 30 s, 1 min, 2 min, 5 min, 10 min, 15 min, or 30 min. Then in the comparison provided in step (c) all values are taken into account the first time stamps of which differ at most by the tolerance value. In this way, it is ensured that minor temporal differences, for example less than 2 s, between the values of different parameters lead to the presence of values for one parameter, but not for another. Alternatively or additionally it can be provided that to each parameter a period of validity is assigned, for example 2 s, 5 s, 10 s, 30 s, 1 min, 2 min, 5 min, 10 min, 15 min, or 30 min, 1 h, 2 h, 1 d, 2 d, etc. Then, these values can be used in step (c) until, as from the first time stamp, the period of validity is expired.

Thus, by the term “same first time stamps” there are understood time stamps (i) being exactly the same time stamps and/or (ii) differing at most by one given tolerance value and/or (iii) lying within the given period of validity.

In one embodiment of the invention step (b) comprises triggering an alert if for one value a second time stamp is present, but not a first time stamp. With this examination it is ensured that all values reaching the system are taken into account in the analysis of the data according to their occurrence time and not to their detection time.

According to the invention, step (d) provides triggering an alert if one of the detected values is outside of a given tolerance range. For that, comparative values and tolerances are given for each parameter that are stored in the system. Preferably, the time of triggering an alert is linked to the alert.

After triggering an alert, there is automatically determined the time at which, as from the third time stamp, a medical measure has to be taken at the latest. The determination of the time is made on the basis of time limits stored in the system. Die length of the time limit may be given for example on the basis of guidelines such as those of the Surviving Sepsis Campaign (SSC) For example, it may be given that a medical measure has to be taken after expiration of 10 min, 20 min, 30 min, 45 min, 60 min, 90 min, 120 min at the latest. If this time limit expires without a medical measure having been taken, preferably there is again triggered an alert.

In order to determine the length of the time limit it can either be provided that after each alert a medical measure has to be taken within a given time limit independent of the parameter(s) that led to the triggering of the alert in step (d). For example, it can be provided that after the triggering of an alert a medical measure has to be taken basically within 1 h. However, it can alternatively be provided to determine the time limit depending on the parameter(s) that led to the triggering of the alert. For that, guidelines are stored in the system in which specific time limits are stored for each parameter or each group of parameters. If, for example the values of the respiratory rate are outside of the tolerance range a time limit of 1 h can be given, whereas for a deviating value of the heart rate a time limit of 20 min can be given.

According to the invention there is further provided the continuous output of the final time determined in step (f) and/or the period left to reach the final time, and/or a time domain being shorter than this period. With this, it is ensured that the physician at all times is aware which period is still left to take a suitable medical measure. The output of the final time or the period until reaching the final time preferably is permanently on a display facility of the system. Alternatively or additionally it can be provided that a time domain is output that is shorter than the period left to reach the final time.

Preferably, in step (g) there are additionally output the value(s) which caused the triggering of the alert in step (d). In this way, it is immediately apparent which of the value(s) caused the triggering of the alert and thus, the determination of the final time, which further simplifies a decision on any medical measure possibly to be taken.

In one embodiment of the invention it can be provided that with output of the alert in step (d) a request for the manual input of information is output, wherein the request is generated on the basis of guidelines stored in the system and wherein further requests for the manual input of information can he output on the basis of the guidelines as soon as a manual input of the requested information is done. In this way, a physician can be led to a decision on the medical measure to be taken in the form of questions what especially proves useful in stressful situations. The requests, i.e. the content of the questions, are generated on the basis of the guidelines as given of example by the Surviving Sepsis Campaign (SSC). Further, it can be provided that each output of a request is associated with the output of the final time determined in step (f) and/or the period left to reach the final time. To make it easier to answer the questions it can further be provided that at least one of the requests for the manual input of information is associated with the output of values of given parameters, wherein it is stored in the system with which parameters the request is associated.

The time domain that is alternatively or additionally output in step (g) preferably starts automatically with the manual input of information after the first output of a request for the manual input of information. Alternatively, a separate request for the start of the time domain can be output on that the time domain only starts if the user wants it. The time domain ends before the final time calculated in step (f). For example, it can be provided that the time domain is 10 min, 20 min, 30 min, 45 min, 60 min, 90 min, 120 min as long as it ends before the final time. If the time limit given in step (f) is for example 6 h and if the alert is triggered at 9:32 o′clock then the final time is 15:32 o′clock. At 9:44 o′clock, the period left to reach the final time is 5 h and 38 min. If a time domain of 1 h is given and the time domain starts automatically with the first manual input or in response to a separate request at 9:44 o′clock then the time domain ends at 10:44 o′clock. The length of the time domain is stored in the system. With that, at the start of the time domain it is examined whether the end of the time domain is before the final time. If this is not the case, the final time and/or the period left to reach the final time are output. If the end of the time domain is before the final time so only the end of the time domain or the time left to reach the end of the time domain are output.

Steps (e) to (g) are not necessarily bound to steps (a) to (d). It is sufficient, if they can be used for each case of triggering an alert. This also applies to the measures described here referring to steps (e) to (g).

In a further embodiment, in step (a) the physiological parameters are assigned to a first group or a second group, wherein the parameters of the first group are continuously determined and the parameters of the second group are determined discontinuously. Here, in the parameters of the first group the first time stamp can be set equal to the second time stamp which is in particular of advantage if the time difference between the first and the second time stamps is less than 10 min, preferably less than 5 min, particularly preferred less than 1 min. In this way, the method according to the invention can be simplified. On the other hand, with parameters of the second group an alert should be triggered if the first time stamp does not differ from the second time stamp since this indicates a false input or manipulation of values or occurrences.

The term “physiological and/or pathological parameter” means a value that (a) indicates the measured value of a measured physiological characteristic of a patient or an indicator (e.g. the oxygen partial pressure in a blood analysis, respiratory rate, body temperature, CKMB concentration, germ load); and (b) can comprise a classification of a physiological condition of a patient (e.g. yellow coloration of the facial skin; presence of a certain germ; existence of a resistance). In one embodiment of the invention an optical or/and audible alert can be triggered if in the detection of values of parameters a given pathogenic germ and/or a resistance of a found germ to a drug that should be prescribed for the patient is found.

The term “indicator” herein relates to compounds or elements that—depending on their type—are produced in biological systems or are introduced in biological systems and the presence or concentration of which (e.g., in a certain organ) is a characteristic for a biological process or a biological condition. For example, such compounds and elements comprise those produced by tumor cells, induced by a tumor in other body cells, and/or changed by a tumor as tumor-specific substances in their concentration. Such indicators are for example macromolecules, e.g. proteins, or trace elements. Such compounds and elements further comprise bone markers that are characteristic of osteoclasis processes such as osteoporosis or enzymes that are important for the assessment of the function of organs.

The term “value” or “measured value” herein relates (a) to all numerical values in the medical field for a parameter that result (e.g. an oxygen partial pressure in a blood gas analysis of 81.2 mmHg, a respiratory rate of 15 breathes per minute, a body temperature of 36.8° C., CKMB concentration of 152 ng/ml; germ load), or (b) to classification results of a physiological condition of a patient (e.g. yellow coloration of the facial skin: no, wherein to classifications by means of statements such as “no” a numerical value, for example “0”, should be assigned).

Preferably, at least the values of one of the physiological parameters that are characteristic of the presence of a disturbance of health are determined using an indicator.

Here, physiological parameters should be selected that due to medical findings are believed to be connected with a certain disturbance of health, for example a respiratory insufficiency. For determining the medical risk of a respiratory insufficiency there are employed for example physiological parameters that are obtained by means of a blood gas analysis. The physiological parameters obtained by means of the blood gas analysis can comprise the pH value of the blood, the blood's oxygen partial pressure, the blood's partial pressure of carbon dioxide, and the oxygen saturation of blood. Further physiological parameters that can be used to assess the medical risk of a respiratory insufficiency in the method according to the invention comprise in addition to the values resulting from the blood gas analysis the respiratory rate (AF) of the patient as well as the age and sex of the patient.

The number of parameters, the values of which are detected in step (a), should be at least 2. If the parameters are assigned to a first and a second group the number of parameters of the first group should be at least 1, preferably at least 2, whereas the number of parameters of the second group independently should be at least 1, preferably at least 2.

A parameter is assigned to the first group if it is detected in a continuous manner. Continuously detecting means the uninterrupted detection of values of the parameter or the detection of values of the parameter in intervals of 24 h or less.

A parameter is assigned to the second group if it is detected in a discontinuous manner. Discontinuous detecting means the single detection of values of the parameter; the detection of values of the parameter in regular or irregular intervals of more than 24 hours; or the complete absence of the detection of values for this parameter if it does not result at any time that the detection of values for said parameter is necessary. For that, the method comprises the continuous determination of the necessity to determine values of parameters of the second group on the basis of previously detected values of parameters of the first group or on the basis of previously detected values of parameters of the first group and the second group.

If it results that a value initially having been assigned to the second group has to be detected in intervals of 24 hours or less so this parameter is classified from the second group into the first group. Such a necessity may for example exist if a parameter initially having been assigned to the second group had been determined in two or more sequential intervals of less than 24 hours. For that, the method according to the invention can comprise detecting the intervals between two sequential determinations of the parameters assigned to the second group and checking whether two or more sequential intervals between detections of the same parameters are 24 hours or less. The number of sequential intervals that are 24 hours or less required for this should be an integer of greater than or equal to 2.

If a parameter is transferred from the second group into the first group an alert can be triggered and/or the category in step (e) can be assigned to said parameter which now is a parameter of the first group.

The term “medical measure” comprises every medical or caring measure that is taken to improve the physical condition of the patient, for example to lead one or more of the detected physiological parameters into a range that is typical of healthy persons.

The taken medical measures can also be detected continuously, i.e. every further measure, every change of an existing measure (e.g. of the dosage of a medicament) and/or the real end of a measure are detected.

In a further embodiment of the method according to the invention an additional step may be provided wherein the completeness of the data is examined. The examination of the completeness of the data may be continuous. Alternatively, it is carried out at expiry of a given period of time, for example every four, eight, sixteen, and/or twenty-four hours. Said time intervals may correspond to the duration of a shift or a working day of the hospital.

The data are considered complete if they comply with the medical guidelines, in particular with the taken medical measures as well as the physiological parameters and other information that have to be detected in accordance to the guidelines of the hospital in general and the guidelines of the physician in particular.

The data that have to be detected in accordance to the medical guidelines are in the following also referred to as “expected data”, since their detection in accordance to the medical guidelines is expected. Single expected data are referred to as “expected indication”.

In order to examine the completeness of the data the actually detected data are compared with data that have to be detected in accordance to the medical guidelines. In that occasion, the detected data are assigned to the expected data. For example, when the detection of the oxygen partial pressure is expected, then the value actually measured and detected for the oxygen partial pressure is assigned to the expected value. Each expected indication can be linked with one or more exact times from which it is evident when a value is expected for the expected indication.

Further, in accordance to the invention a system for monitoring the medical condition of a large number of patients according to the method of the present invention is provided. The system comprises a processor, a memory, an input device, and a display device, wherein
the input device enables the user to manually input information for each patient;
in the memory the detected values of parameters as well as the first and second time stamps for each of these patients are stored;
the processor carries out the continuous comparison of values of different parameters having the same first time stamp to comparative values stored in the system; the triggering of an alert if the comparison shows that at least one of the values is outside of a given tolerance range with respect to the comparative value; the linkage of the alert to a third time stamp representing the time of the triggering of the alert; and the determination of the time (final time) as from the third time stamp at which a medical measure has to be taken at the latest, wherein the determination of the time is based on the basis of given time limits stored in the system; and
the display device continuously outputs the final time determined in step (f) and/or the period left to reach the final time or the time domain.

Furthermore, in the memory there can be stored data bases for the guidelines, the comparative values and their tolerance ranges.

Furthermore, the system can comprise a device for examining the completeness of the data.

The system can be a computer-implemented system, in particular a computer-implemented patient data management and/or decision assist system.

In the following the invention is explained in detail with respect to the drawings. Here,

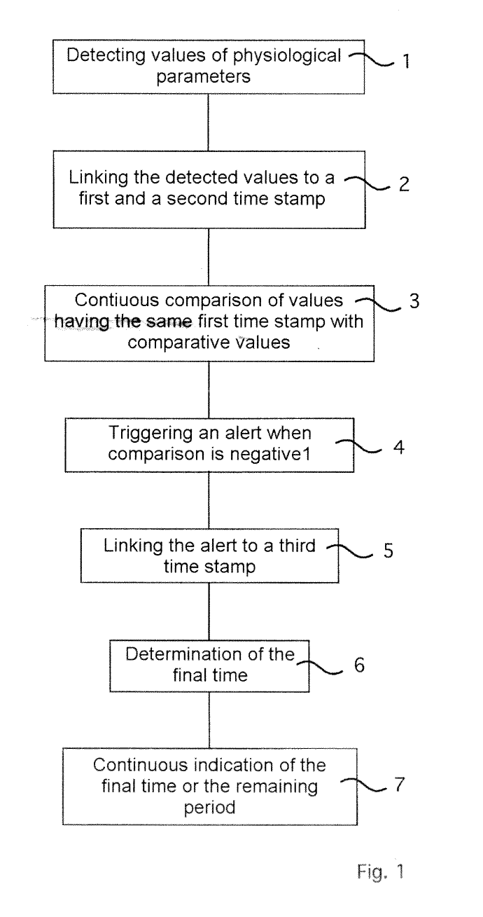

FIG. 1 shows a schematic representation of an embodiment of the method according to the invention;

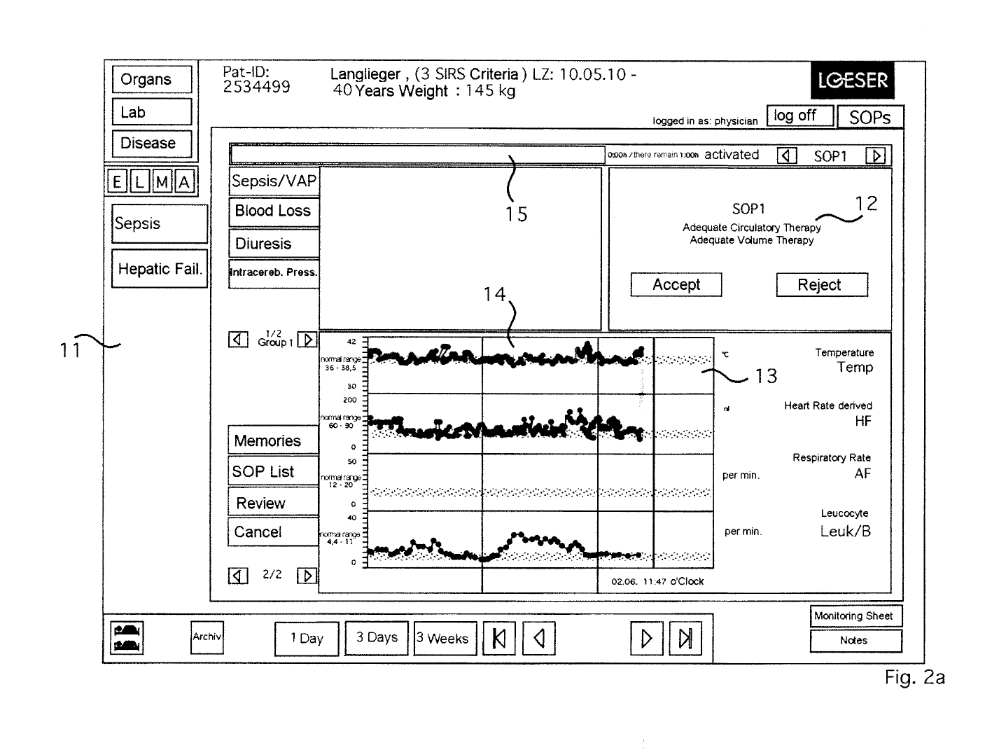

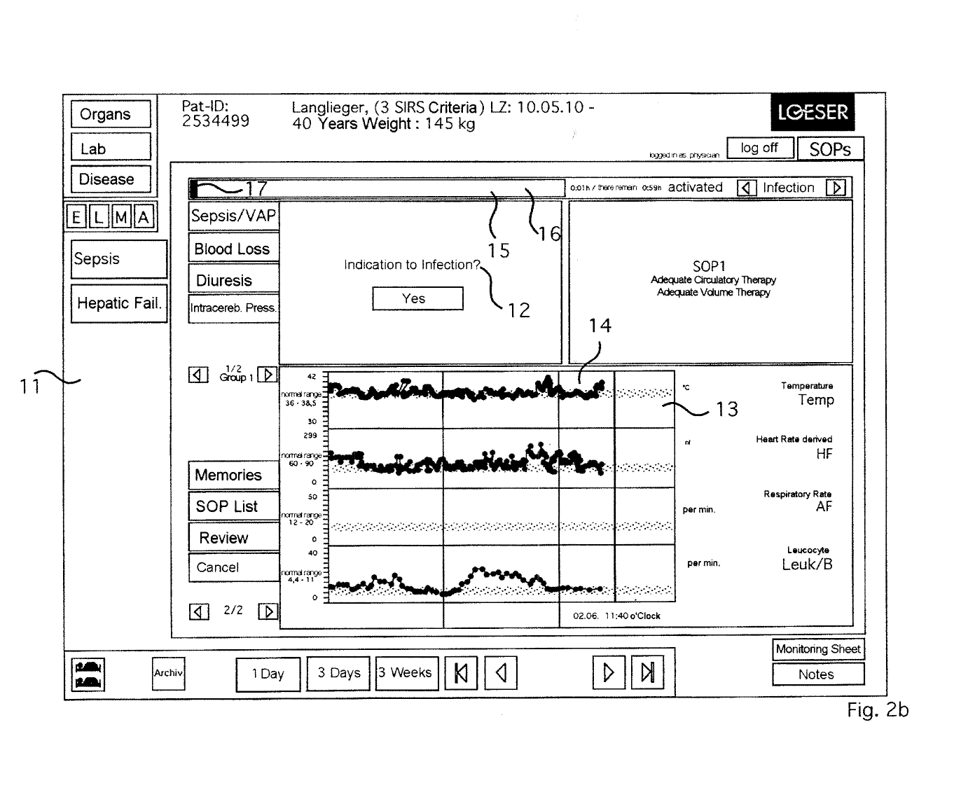

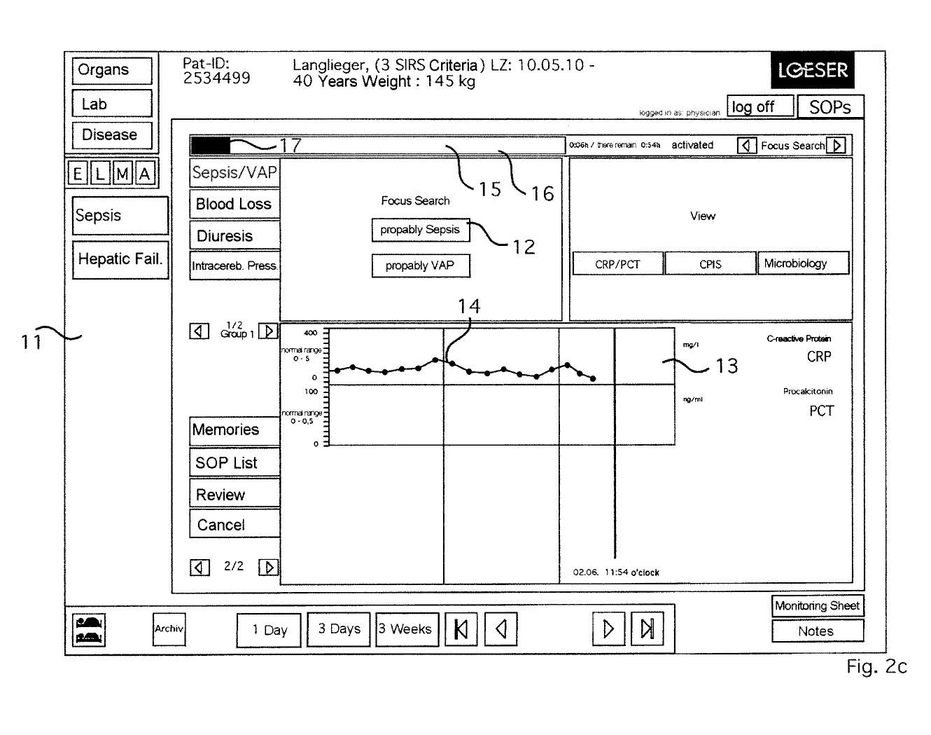

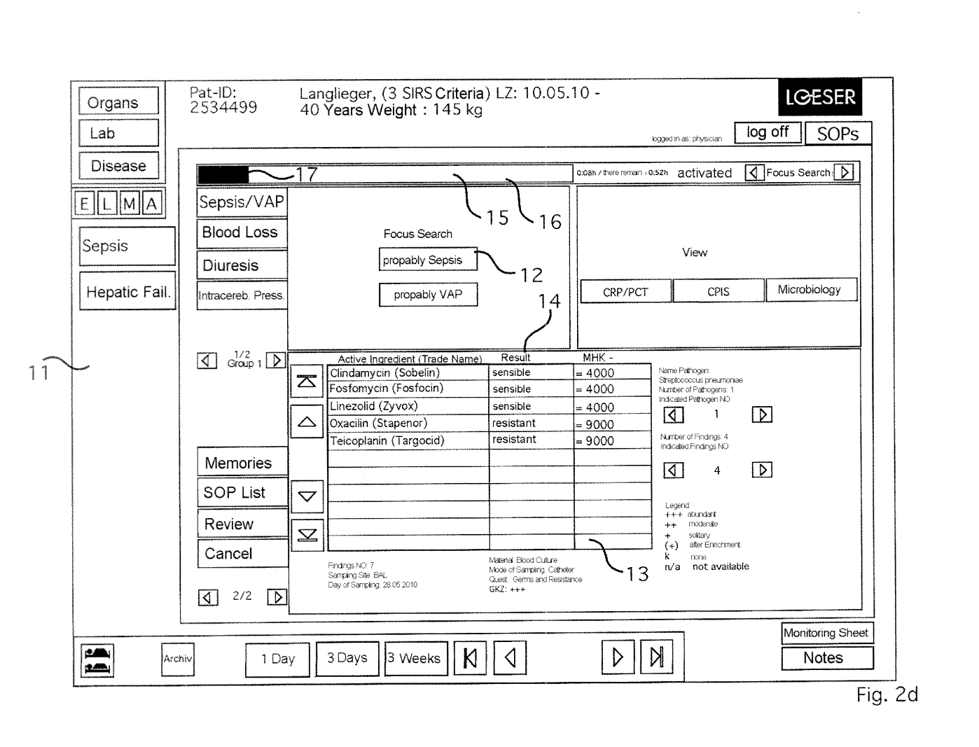

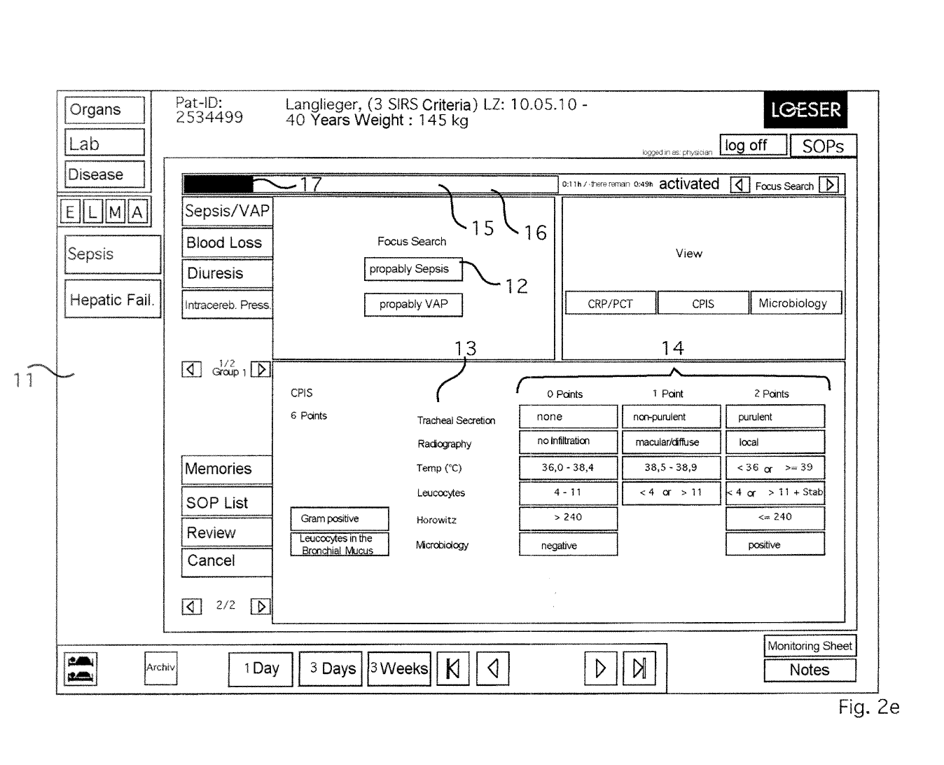

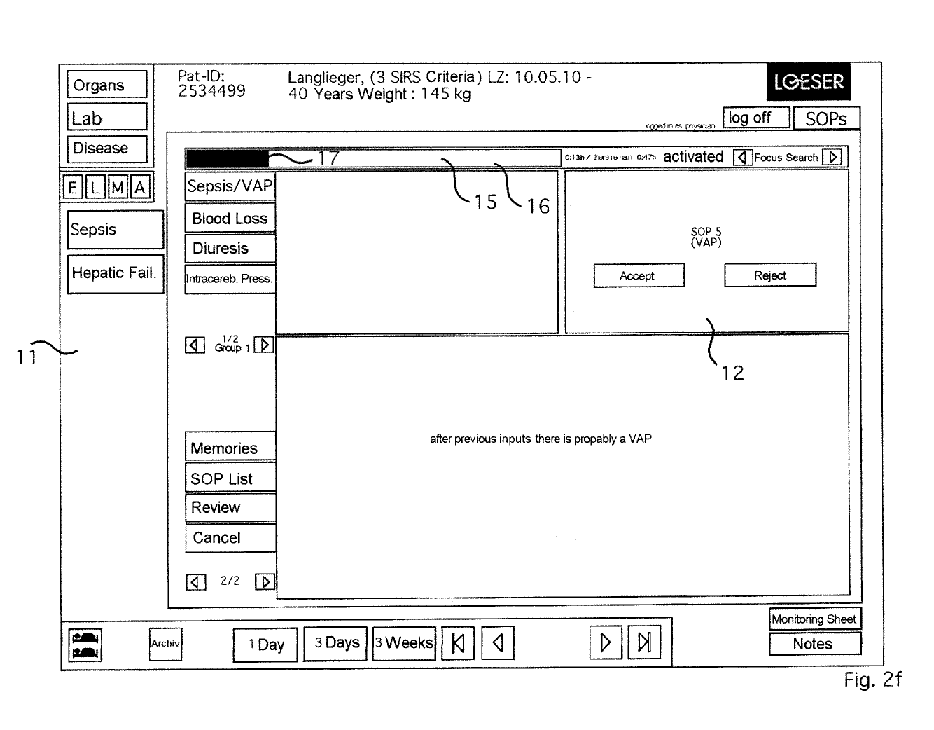

FIG. 2a-e show schematic representations of screen images of the display device according to the invention:

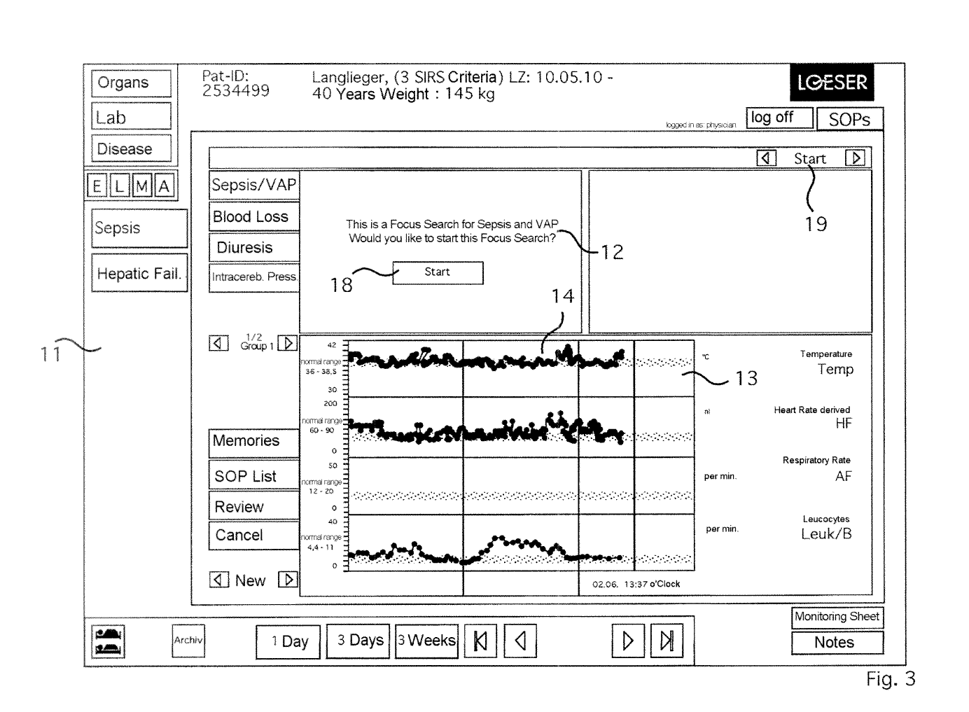

FIG. 3 shows a further schematic representation of a screen image of the display device according to the invention.

In the embodiment of the method according to the invention shown in FIG. 1 at first continuously or discontinuously values of physiological parameters of the patient are detected, at least a part of which are characteristic for the presence of a disturbance of health 1. The detected values are linked to a first time stamp and a second time stamp, wherein the first time stamp represents the time of occurrence of the value and the second time stamp represents the time of detection of the value 2. Subsequently, the values of all parameters having the same first time stamp are continuously compared to comparative values stored in the system 3. A same time stamp of the value of a first parameter can be present in a proportion to the value of a second (or any other) parameter if (i) the value of the first parameter and the value of the second parameter have exactly the same time stamp, or if (ii) the value of the first parameter at most differs from the value of the second parameter by a given tolerance value, or if (iii) the value of the first parameter is within a given period of validity as from its first time stamp and the first time stamp of the second parameter is also within the period of validity of the value of the first parameter, or the first time stamp of the second parameter only differs by the given tolerance value from the period of validity of the value of the first parameter, or if (iv) the value of the second parameter is within the given period of validity as from its first time stamp and the first time stamp of the first parameter is also within the period of validity of the value of the second parameter or the first time stamp of the first parameter only differs by the given tolerance value from the period of validity of the value of the second parameter.

If the comparison in step (c) shows that at least one of the values is outside of a given tolerance range with respect to the comparative value, so an alert is triggered 4. Here, the alert is linked to a third time stamp representing the time of the triggering of the alert 5. Thereafter, the time (final time) as from the third time stamp is determined at which a medical measure has to be taken at the latest 6. Determination of the time is made on the basis of given time limits stored in the system. The thus established final time and/or the period left to reach the final time are then output on the display device of the system.

Together with the output of the alert several requests for the manual input of information can successively be output on the display device that have been generated on the basis of guidelines stored in the system. In FIG. 2a-e screen images 11 of the display device are shown on which such requests 12 are represented together with values 14 of parameters 13 that should facilitate the user the input of the requested information. Moreover, the period left to reach the final time is shown in the form of a status bar 15 (FIG. 2a). In the following FIGS. 2b to 2f the remaining period 16 is respectively represented colored separate from the period 17 already elapsed since the triggering of the alert. If the user enters the requested information as represented in the order of the figures, so the decision assist system finally generates a proposal which can help the user to find a diagnosis and select a suitable medical measure (FIG. 2f).

In FIG. 3 a screen image is shown with which a first request for the manual input of data is output. Here, the beginning of the time domain can be started automatically with the manual input of information by pressing soft key 18, or alternatively by pressing soft key 19 if no automatic start is intended. If now the end of the time domain and the time until the end of the time domain are output on the display device, so the screen images displayed to the user correspond to the screen images shown in FIGS. 2a to 2f except that instead of the final time and the period left to reach the final time the end of the time domain and the time left to the end of the time domain are represented.

1. A method for monitoring the medical condition of a patient by means of a patient data management and/or decision assist system comprising the steps of
(a) detecting values of physiological parameters of the patient, wherein at least a part of the physiological parameters characterizes the presence of a disturbance of health;
(b) linking the detected values to a first time stamp and a second time stamp, wherein the first time stamp indicates the time of occurrence of the value and the second time stamp the time of detection of the value;
(c) continuously comparing the values of different parameters having the same first time stamp with comparative values stored in the system;
(d) triggering an alert if the comparison shows that at least one of the values is outside of the given tolerance range with respect to the comparative value;
(e) linking the alert to a third time stamp indicating the time of triggering the alert;
(f) determining the time (final time) as from the third time stamp at which a medical measure must be taken at the latest, wherein the determination of the time is made on the basis of given time limits stored in the system; and
(g) continuously outputting (i) the final time determined in step (f) and/or (ii) the period left to reach the final time and/or (iii) a time domain being shorter than the period left to reach the final time.

2. The method according to claim 1, characterized in that in step (g) there are additionally output the value(s) that caused the triggering of the alert in step (d).

3. The method according to claim 1, characterized in that step (b) comprises the triggering of an alert if for one value a second time stamp is present, but not a first time stamp.

4. The method according to claim 1, characterized in that in step (a) the physiological parameters are assigned to a first group or a second group, wherein the parameters of the first group are continuously determined and the parameters of the second group are determined discontinuously, wherein in the parameters of the first group the first time stamp is set equal to the second time stamp, and/or wherein in parameters of the second group an alert should be triggered if the first time stamp does not differ from the second time stamp.

5. The method according to claim 1, characterized in that with the output of the alert in step (d) a request for the manual input of information is output, wherein the request is generated on the basis of guidelines stored in the system and wherein further requests for the manual input of information can be output on the basis of the guidelines as soon as a manual input of the requested information is done.

6. The method according to claim 1, characterized in that each output of a request is associated with the output of the final time determined in step (f) and/or the period left to reach the final time.

7. The method according to claim 5, characterized in that at least one of the requests for the manual input of information is associated with the output of values of given parameters, wherein it is stored in the system with which parameters the request is associated.

8. A system for monitoring the medical condition of a large number of patients comprising a processor, a memory, an input device, and a display device, wherein
the input device enables the user to manually input information for each patient;
in the memory the detected values of parameters as well as the first and second time stamps for each of these patients are stored;
the processor carries out the continuous comparison of values of different parameters having the same first time stamp to comparative values stored in the system; the triggering of an alert if the comparison shows that at least one of the values is outside of a given tolerance range with respect to the comparative value; the linkage of the alert to a third time stamp representing the time of the triggering of the alert; and the determination of the time (final time) as from the third time stamp at which a medical measure has to be taken at the latest, wherein the determination of the time is based on the basis of given time limits stored in the system; and
the display device continuously outputs the final time determined in step (f) and/or the period left to reach the final time or the time domain.

9. The method according to claim 2, characterized in that step (b) comprises the triggering of an alert if for one value a second time stamp is present, but not a first time stamp.

10. The method according to claim 2, characterized in that in step (a) the physiological parameters are assigned to a first group or a second group, wherein the parameters of the first group are continuously determined and the parameters of the second group are determined discontinuously, wherein in the parameters of the first group the first time stamp is set equal to the second time stamp, and/or wherein in parameters of the second group an alert should be triggered if the first time stamp does not differ from the second time stamp.

11. The method according to claim 3, characterized in that in step (a) the physiological parameters are assigned to a first group or a second group, wherein the parameters of the first group are continuously determined and the parameters of the second group are determined discontinuously, wherein in the parameters of the first group the first time stamp is set equal to the second time stamp, and/or wherein in parameters of the second group an alert should be triggered if the first time stamp does not differ from the second time stamp.

12. The method according to claim 9, characterized in that in step (a) the physiological parameters are assigned to a first group or a second group, wherein the parameters of the first group are continuously determined and the parameters of the second group are determined discontinuously, wherein in the parameters of the first group the first time stamp is set equal to the second time stamp, and/or wherein in parameters of the second group an alert should be triggered if the first time stamp does not differ from the second time stamp.

13. The method according to claim 2, characterized in that with the output of the alert in step (d) a request for the manual input of information is output, wherein the request is generated on the basis of guidelines stored in the system and wherein further requests for the manual input of information can be output on the basis of the guidelines as soon as a manual input of the requested information is done.

14. The method according to claim 3, characterized in that with the output of the alert in step (d) a request for the manual input of information is output, wherein the request is generated on the basis of guidelines stored in the system and wherein further requests for the manual input of information can be output on the basis of the guidelines as soon as a manual input of the requested information is done.

15. The method according to claim 4, characterized in that with the output of the alert in step (d) a request for the manual input of information is output, wherein the request is generated on the basis of guidelines stored in the system and wherein further requests for the manual input of information can be output on the basis of the guidelines as soon as a manual input of the requested information is done.

16. The method according to claim 9, characterized in that with the output of the alert in step (d) a request for the manual input of information is output, wherein the request is generated on the basis of guidelines stored in the system and wherein further requests for the manual input of information can be output on the basis of the guidelines as soon as a manual input of the requested information is done.

17. The method according to claim 2, characterized in that each output of a request is associated with the output of the final time determined in step (f) and/or the period left to reach the final time.

18. The method according to claim 3, characterized in that each output of a request is associated with the output of the final time determined in step (f) and/or the period left to reach the final time.

19. The method according to claim 4, characterized in that each output of a request is associated with the output of the final time determined in step (f) and/or the period left to reach the final time.

20. The method according to claim 5, characterized in that each output of a request is associated with the output of the final time determined in step (f) and/or the period left to reach the final time.
